(19) United States
(12) Patent Application Publication
(10) Pub. No.: US 2015/0238196 A1
(43) Pub. Date: Aug. 27, 2015

(54) ELONGATE EXPANDABLE MEMBER FOR OCCLUDING VASCULAR VESSEL

(71) Applicant: Vascular Solutions, Inc., Minneapolis, MN (US)

(72) Inventors: Howard Root, Excelsior, MN (US); James Arnold Murto, Maple Grove, MN (US); Stephen Anthony Penegor, Watertown, MN (US)

(21) Appl. No.: 14/630,291

(22) Filed: Feb. 24, 2015

Related U.S. Application Data
(60) Provisional application No. 61/945,699, filed on Feb. 27, 2014.

(51) Int. Cl. A61B 17/12

(52) U.S. Cl. CPC A61B 17/12109; A61B 17/1204

(57) ABSTRACT

Assemblies and methods for occluding a vascular vessel are disclosed. An assembly can include a removable outer member, an elongate expandable member, a porous cover member, and optionally, one or both of a removable inner tubular member or a seal member. The elongate expandable member and the porous cover member can be positioned in a radially compressed form between the removable outer members and, if present, the inner tubular member, with the porous cover member surrounding an outer surface of the elongate expandable member. The elongate expandable member can be configured to expand the porous cover member and occlude a vascular vessel following removal of the removable outer member. Mechanical stability and migration resistance of the elongate expandable member can be aided by the porous cover member. The seal member can be positioned at a distal end of the elongate expandable member to inhibit its expansion during vessel insertion.

### CLAIM OF PRIORITY

This non-provisional patent application claims the benefit of priority under 35 U.S.C. §119(e) to U.S. Provisional Patent Application Ser. No. 61/945,699, entitled “ELONGATE EXPANDABLE MEMBER FOR OCCLUDING VASCULAR VESSEL,” (Attorney Docket No. 3195.080PRV), filed on Feb. 27, 2014, which is herein incorporated by reference in its entirety.

### BACKGROUND

Vascular vessels are the conduits for circulating blood through a mammalian body. A vascular vessel that carries blood away from a heart is known as an artery. A vascular vessel that returns blood to the heart is known as a vein.

To assist blood flow, veins include venous valves. Each venous valve is located inside a vein and typically includes at least two valve leaflets that are disposed annularly along inside walls of the vein. These valve leaflets open to permit blood flow toward the heart and close, upon a change in pressure, to restrict the retrograde flow (or reflux) of blood. When blood flows toward the heart, venous pressure forces the valve leaflets to move apart in a downstream flexing motion and create an open path for blood flow. The leaflets normally return to a closed position to restrict or prevent blood flow in a retrograde direction after the venous pressure is relieved. The leaflets, when functioning properly, extend radially inward toward one another such that leaflet tips contact each other when the valve is closed.

On occasion, and for a variety of reasons including congenital valve or vein weakness, disease in the vein, obesity, pregnancy, or an occupation requiring long periods of standing or sitting, one or more valves in a vein may allow retrograde blood flow to occur. When a valve allows such retrograde flow, blood can collect in one or more vascular vessels beneath the valve and cause an increase in the venous pressure there. Venous valves that allow retrograde flow are known as incompetent venous valves. Incompetent venous valves can cause swelling in the patient's lower extremities and veins to bulge, resulting in varicose veins. If left untreated, varicose veins can cause feelings of aching, pain, leg heaviness and fatigue, and can further cause aesthetic issues.

Surgical and non-surgical methods for treatment of varicose veins exist. An example non-surgical method for treatment of varicose veins is the placement of an elastic stocking around a patient's leg. The stocking can apply external pressure to the vein, forcing the vein walls radially inward and the leaflets into apposition. Another non-surgical treatment method is sclerotherapy, which involves the direct injection of a sclerosing or other chemical solution along the length of the varicose vein using a needle. The chemical solution can irritate the vein tissue, causing the lining of the vein to swell, harden, and eventually seal off. An example surgical method for treatment of varicose veins includes bringing incompetent leaflets into closer proximity, in hopes of restoring natural valve function, by implanting a frame around the outside of the vessel, placing a constricting suture around the vessel, or other types of treatment to the outside of the vessel to induce vessel contraction. Other surgical treatment methods include bypassing or replacing damaged venous valves with autologous sections of veins containing competent valves and vein stripping and ligation.

More recently, a number of methods have been suggested to treat varicose veins and venous valve leaflets with energy sources, such as radiofrequency (RF) or laser energy. In one such method, valve leaflets can be fastened together with electrodes delivering RF energy. In another such method, a catheter or laser fiber having an electrode tip can be used to apply RF or laser energy to venous wall tissue causing localized heating and corresponding tissue destruction. After treatment of one venous wall section is complete, the catheter or laser fiber can be repositioned to treat a different venous wall section.

### Overview

The present inventors recognize, among other things, that existing varicose vein treatments are associated with limitations and drawbacks. For example, an elastic stocking placed around a patient's leg can be uncomfortable, especially in warm weather, and the stocking must be constantly worn to keep venous valve leaflets in apposition. The elastic stocking can also affect the patient's physical appearance, potentially having an adverse psychological effect on him/her leading to removal of the stocking. Sclerotherapy can result in patient swelling due to agent injection and numerous needle pokes. Vein bypassing and vein stripping and ligation require at least one incision, are associated with a relatively long patient recovery time and bruising, have the potential for scarring, and numerous other risks inherent with surgery, such as those associated with the administration of systemic anesthesia. Application of RF or laser energy requires expensive capital equipment (e.g., an energy source), vein insulation due to heat dangers, compression means and a dialing-in of energy, and can cause thermal or perforation damage to a vessel of the patient.

The present assemblies and methods provide a varicose vein treatment associated with minimal patient discomfort, bruising and risk of vessel perforation, does not require an investment in capital equipment or thermal insulation, and can be completed in a relatively fast manner without requiring blood extravasation. The treatment components are mechanically stable, resist migration, and leave behind a cosmetically pleasing implant.

An example assembly can include a removable inner tubular member, a removable outer member, an elongate expandable member, and a porous cover member. The elongate expandable member and the porous cover member can be positioned in a radially compressed form between the removable outer member and the removable inner tubular member, with the porous cover member surrounding an outer surface of the elongate expandable member. The elongate expandable member can be configured to expand the porous cover member and occlude a vascular vessel following removal of the outer member. To facilitate their removal, one or both of the inner or outer members can include a handle or hub coupled to their proximal ends. Mechanical stability and migration resistance of the elongate expandable member can be aided by the porous cover member, such as by securing a proximal portion of the porous cover member to subcutaneous tissue. The assembly can optionally further include a seal member positioned at a distal end of the elongate expandable member.

An example method can include inserting an elongate expandable member and a porous cover member into a vascular vessel. The elongate expandable member and the porous cover member can be radially compressed, optionally about a removable inner tubular member, enclosed around their respective outer surfaces by a removable outer member, and optionally distally sealed by a seal member. The elongate expandable member and the porous cover can be advanced through the vascular vessel by guiding a lumen of the removable inner tubular member along a guidewire. The outer member can be removed to allow the elongate expandable member and the porous cover member to expand from a radially compressed first diametrical size or first cross-sectional area to a second larger diametrical size or second larger cross-sectional area and occlude the vascular vessel.

These and other examples and features of the present assemblies and methods will be set forth in part in the following Detailed Description. This Overview is intended to provide non-limiting examples of the present subject matter—it is not intended to provide an exclusive or exhaustive explanation. The Detailed Description below is included to provide further information about the present assemblies and methods.

### BRIEF DESCRIPTION OF THE DRAWINGS

In the drawings, like numerals can be used to describe similar features and components throughout the several views. The drawings illustrate generally, by way of example but not by way of limitation, various embodiments discussed in the present patent document.

FIGS. 1-2 illustrate vessel structures of a human leg, which provide suitable environments for use of the present assemblies and methods, as constructed in accordance with at least one embodiment.

FIG. 3 illustrates an isometric view of an assembly for occluding a vascular vessel, as constructed in accordance with at least one embodiment.

FIG. 4 illustrates a method of using an assembly for occluding a vascular vessel, as constructed in accordance with at least one embodiment.

FIG. 5 illustrates an elongate expandable member, a porous cover member, and optionally a seal member of an assembly located in, and occluding, portions of a great saphenous vein, as constructed in accordance with at least one embodiment.

FIG. 6 illustrates a method of manufacturing an assembly for occluding a vascular vessel, as constructed in accordance with at least one embodiment.

FIG. 7 illustrates a proximal end view of an assembly for occluding a vascular vessel, as constructed in accordance with at least one embodiment.

FIG. 8 illustrates a side cross-sectional view of an assembly for occluding a vascular vessel, as constructed in accordance with at least one embodiment.

FIG. 9 illustrates a side cross-sectional view of a distal portion of an assembly for occluding a vascular vessel, as constructed in accordance with at least one embodiment.

FIGS. 10-14 illustrate seal members that can optionally be positioned at a distal end of an elongate expandable member to inhibit expansion of the expandable member until an operator-elected time period, as constructed in accordance with certain embodiments.

FIGS. 15-16 illustrate porous cover members that can be positioned around an outer surface of an elongate expandable member to support its longitudinal integrity and inhibit its migration, as constructed in accordance with certain embodiments.

FIGS. 17A-C schematically illustrate the gradual absorption and/or degradation of an elongate expandable member, a porous cover member, and an optional seal member within a vascular vessel, as constructed in accordance with at least one embodiment.

The drawing figures are not necessarily to scale. Certain features and components may be shown exaggerated in scale or in schematic form and some details may not be shown in the interest of clarity and conciseness.

### DETAILED DESCRIPTION

Varicose veins are quite common for both men and women. In fact, tens of millions of people in the U.S. have varicose veins—with 50% of the population age 50 and older suffering from varicose veins. Some risk factors related to the manifestation of varicose veins include age, heredity, gender, obesity, pregnancy, and prolonged standing or sitting. Symptoms related to varicose veins can vary from mild to severe with aching, pain, leg heaviness and swelling, fatigue, and aesthetic issues varying based on the severity of the disease. More severe symptoms can include deep vein thrombosis, pulmonary embolism, and ulceration, which can lead to serious health problems and even death if left untreated.

The present inventors recognize that the treatment of varicose veins is important, and further recognize that existing varicose vein treatment assemblies and methods are associated with limitations and drawbacks. Unlike existing treatments, the present assemblies and methods do not involve heating of the treated vascular vessel and, therefore, do not pose any risk of thermal damage to the sensory nerves associated with the vessel. The present assemblies provide an occlusive elongate expandable member and a porous cover member that can be implanted using a relatively simple and quick method, which is easy to perform, can be done under local anesthesia, and induces minimal postoperative pain.

The elongate expandable member can include a sponge- or foam-like structure and the porous cover member can include a knitted, woven or braided structure so that the assembly is longitudinally flexible and radially compact during insertion. Together, the longitudinal flexibility and radial compactness of the assembly permit easy insertion into a vascular vessel to be treated and do not generate any appreciable rigidity under a patient's skin post-implant. The sponge- or foam-like structure of the elongate expandable member can ensure reliable occlusion of the vascular vessel resulting in thrombosis of the vessel, which gradually organizes into fibrous tissue. Mechanical stability and migration resistance of the elongate expandable member can be aided by the porous cover member, such as by securing a proximal portion of the porous cover member to subcutaneous tissue.

The assemblies and methods can be used for a wide range of indications, including the treatment of varicosities associated with superficial reflux of a great or lesser saphenous vein, a branch superficial or perforator vein, or other veins or arteries in the legs or elsewhere in the body. Further, biliary ducts, ureteral vessels, alimentary canals, or other body passages may find benefit from the present assemblies and methods.

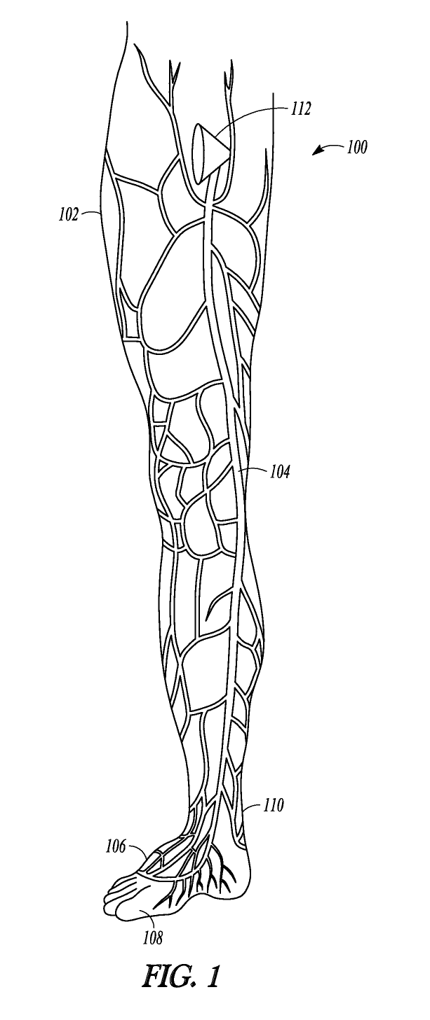

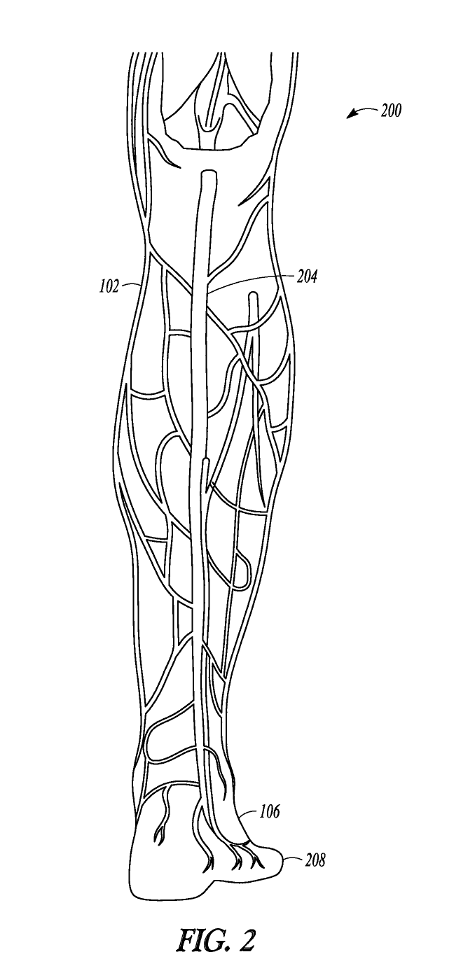

FIGS. 1 and 2 illustrate vascular vessel structures 100, 200 of a human leg 102, which provide suitable environments for use of the present assemblies and methods. Among other things, FIG. 1 illustrates a great saphenous vein 104, which is a large superficial vein on an anterior side of the leg 102. The great saphenous vein 104 originates from where the dorsal vein of a large toe 108 merges with the dorsal venous arch of a foot 106. After passing anterior to a medial malleolus 110, the vein 104 runs up a medial side of the leg 102. At the knee, the great saphenous vein 104 runs over the femur bone and then extends medially on an anterior surface of the thigh until it joins with a femoral vein 112.

FIG. 2 illustrates a lesser saphenous vein 204, which is a large superficial vein on a posterior side of the leg 102. The lesser saphenous vein 204 originates from where the dorsal vein of a smallest toe 208 merges with the dorsal venous arch of the foot 106. The lesser saphenous vein 204 runs along the posterior surface of the leg 102, passes between heads of the gastrocnemius muscle, and drains into the popliteal vein at or above the knee joint.

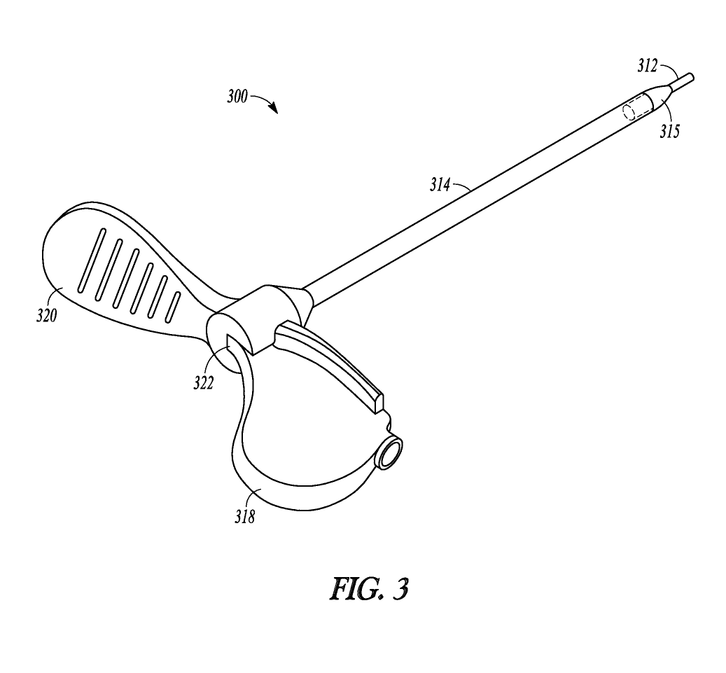

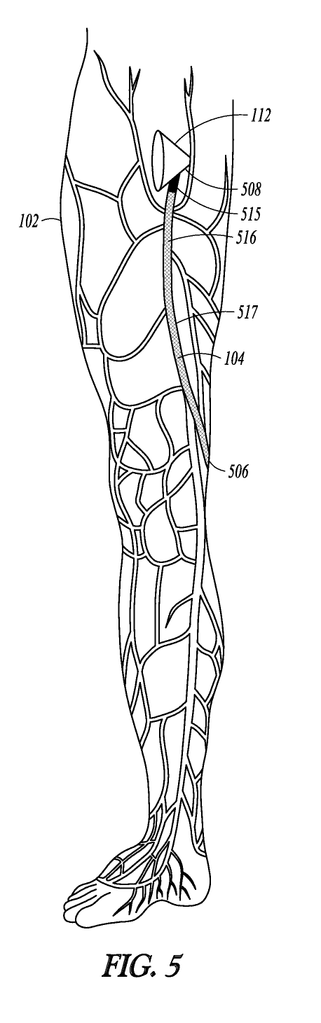

In accordance with the present assemblies and methods, one or more portions of a branch superficial or perforator vein, the lesser saphenous vein 204, or the great saphenous vein 104 can be occluded. Desirably, the occlusion can be effective to prevent reflux of venous blood in a downward direction, thereby treating varicosities that commonly occur in lower portions of the leg 102. With reference to FIGS. 3 and 5, occlusion of a portion of the great saphenous vein 104 can be achieved by deploying an elongate expandable member surrounded by a porous cover member and optionally sealed at a distal end by a seal member into the vein 104. The elongate expandable member and the porous cover member can be initially positioned in a radially compressed or compacted form between portions of a removable inner tubular member and a removable outer member of an assembly.

FIG. 3 illustrates an isometric view of an assembly 300 for occluding a vascular vessel, such as a great or lesser saphenous vein or a branch superficial or perforator vein, as constructed in accordance with at least one embodiment. The assembly 300 can comprise a removable inner tubular member 312, a removable outer member 314, an elongate expandable member, a porous cover member, and a seal member 315. The elongate expandable member and the porous cover member can be positioned in a radially compressed form between portions of the removable inner tubular member 312 and the removable outer member 314. The seal member 315, which is partially shown in phantom, can be positioned at a distal end of the elongate expandable member and partially surrounded by the removable outer member 314. The removable inner tubular member 312 can include a polyimide material and the removable outer member 314 can include an impermeable polytetrafluoroethylene or polyimide material optionally reinforced by a coil or braid member. The reinforcing coil or braid member can provide radial strength to the removable outer member 314, thereby keeping its inner diameter circular and open when delivering the assembly 300 in a tortuous setting. The removable inner tubular member 312 and the removable outer member 314 can each include a handle or hub 318 and 320, respectively, coupled to their proximal ends. Adjacent inner surfaces of the handles or hubs 318 and 320 can form a snap-fit connection 322. The snap-fit connection 322 can be separated when desired by an operator.

The elongate expandable member can be configured to expand and occlude a vascular vessel following in situ removal of the outer member 314. The elongate expandable member includes a structure and composition configured to be radially compressed for insertion into the vascular vessel and, following removal of the outer member 314, can allow for absorption of vessel fluid such as blood. The intake of fluid causes the elongate expandable member to expand and occlude the flow of fluid through the vessel.

The porous cover member can be positioned around an outer surface of the elongate expandable member to support its longitudinal integrity, which can be particularly useful after the inner tubular member 312 and the outer member 314 are removed in situ. The porous cover member allows blood or other fluid to flow into and expand the elongate expandable member.

The optional distally-positioned seal member 315 can inhibit or prevent expansion of a distal portion of the elongate expandable member until an operator-elected time period when the outer member 314 is removed. A distal tip of the seal member 315 can include a rounded or tapered portion designed to be atraumatic to a vessel wall or other subcutaneous tissue during insertion of the assembly 300. The seal member 315 can include a lumen through which the removable inner tubular member 312 extends for receipt of a guidewire.

The elongate expandable member, the porous cover member, and/or the seal member 315 can be composed of one or more bioabsorbable or biodegradable materials that are effective to promote or receive the in-growth of patient tissue. Several bioabsorbable or biodegradable materials are approved for use by the U.S. Food and Drug Administration (FDA) and suitable for use in the assembly 300. Example materials include polyglycolic acid (PGA), polylactic acid (PLA), polyglactin (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials bioabsorb or biodegrade in vivo in a matter of weeks or months, although some more crystalline forms can bioabsorb or biodegrade more slowly. The bioabsorbable or biodegradable materials utilized in the elongate expandable member and the porous cover member can be configured to gradually dissipate after implantation, independent of the mechanism(s) by which dissipation can occur, such as dissolution, degradation, absorption, or excretion. The terms bioabsorption, biodegradation and similar can be used interchangeably and refer to the ability of the material or its degradation products to be absorbed or removed by biological events, such as by enzymatic activity, cellular activity, or fluid transport away from the site of implantation. Accordingly, both bioabsorbable and biodegradable terms are used in this patent document to encompass absorbable, bioabsorbable, and biodegradable, without implying the exclusion of the other classes of materials.

If desirable for a given application, one or both of the porous cover member or the seal member 315 can include a non-bioabsorbable or non-biodegradable material. The non-bioabsorbable or non-biodegradable material can be a permanent implant within a patient's vessel.

Optionally, one or both of the elongate expandable member or the porous cover member can include antibiotics, thrombus-promoting substances (e.g., thrombin or fibrinogen), growth factors, tissue attachment factors, or other active agents having the property of promoting cellular invasion and tissue in-growth. The active agents can be applied onto one or both of the elongate expandable member or the porous cover member via contact with a solution or suspension of the active agent, for example by spraying, dipping, and so forth, followed by evaporation of the solution or suspension's liquid component. The active agent can also be incorporated during the processing or shaping of the material(s) used to form the elongate expandable member or the porous cover member.

One or both of the elongate expandable member or the porous cover member can additionally or alternatively include an antimicrobial agent selected from, for example, silver compounds, chlorhexidine, antibiotics, iodine-containing agents, and certain heavy metals. In an example, a silver compound in the form of silver chloride can be selected as the antimicrobial agent. In the presence of blood, ionic silver can be released from the silver chloride to prevent microorganisms from colonizing on the elongate expandable member or the porous cover member. Ionic silver, an atom of silver that is missing one electron, can provide the antimicrobial property by altering the protein structure and preventing bacterial cells from carrying out normal functions.

A kit can comprise the assembly 300, a needle (e.g., a 21G needle), a guidewire (e.g., an 0.018 in guidewire), and instructions for using the assembly 300 to insert the elongate expandable member, the porous cover member, and optionally the seal member 315 within a vascular vessel such as a great (FIG. 1) or lesser (FIG. 2) saphenous vein or a branch superficial or perforator vein. The elongate expandable member can have a variety of lengths sufficient to achieve occlusion of the desired stretch of vessel, such as about 10 centimeters (cm), about 25 cm, about 50 cm, or about 75 cm and longer. In an example, the elongate expandable member can have a shorter length, such as about 1 cm or 2 cm for occlusion of a branch superficial or perforator vein. In examples where the assembly 300 has low pushability or column strength, an introducer sheath of appropriate size (e.g., a 7F tear-away introducer sheath with a dilator designed for placement over the guidewire) can be included in the kit and used to deploy the assembly 300 using an over-the-guidewire method.

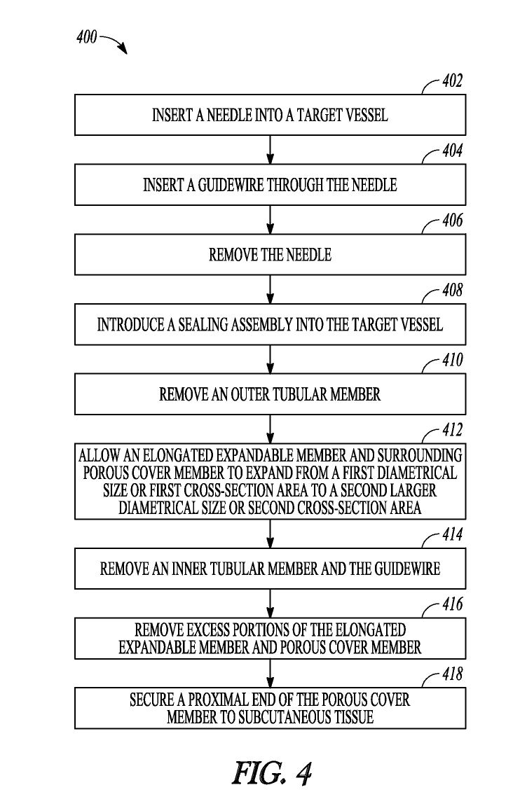

FIG. 4 illustrates an example method 400 of using components of the kit, including the present assembly 300, for occluding a vascular vessel. The method can be implemented under local anesthesia by first inserting a needle into a target vascular vessel in operation 402. A guidewire, such as a 0.018 inch (in) guidewire, can then be inserted through an inner lumen of the needle in operation 404 and into the target vessel, thereby providing a “railway” to the vessel. Once the guidewire is in place, the needle can be removed in operation 406.

The assembly can be introduced into the target vessel in operation 408 using an over-the-guidewire technique through an appropriately sized introducer sheath, with the guidewire passing through a lumen of the removable inner tubular member. Portions of the assembly, particularly the elongate expandable member, the porous cover member, and the optional distally-positioned seal member, can be sufficiently radially compressed or sized so that their outer diameters are smaller than the lumen of the introducer sheath for ease of insertion. Ultrasound or x-ray techniques can be used to visualize a distal tip of the assembly during vessel introduction, such as for monitoring an implanted depth of the assembly.

Once introduced into the target vessel, the removable outer member can be removed in operation 410. Removal of the outer member can include separating an engagement between an outer surface of the seal member and an inner surface of the removable outer member, while preserving an engagement between the outer surface of the seal member and an inner surface of the porous cover member. In an example, a handle or hub attached to a proximal end of the removable inner tubular member can be held in place while a handle or hub attached to a proximal end of the removable outer member is moved in a direction away from removable inner tubular member's handle or hub. This relative handle or hub movement can cause a blade integrated in the removable inner tubular member's handle or hub to contact and cut the removable outer member in a proximal-to-distal direction. In an alternative example, proximal pulling of a cutting wire, which is positioned between the porous cover member and the removable outer member, can allow for separation of the outer member in a distal-to-proximal direction.

With the outer member removed, the removable inner tubular member, the guidewire, the elongate expandable member, the porous cover member, and the seal member remain. The elongate expandable member is now free to absorb vessel fluid and expand to occlude the flow of fluid, such as blood, through the vessel in operation 412. In some examples, the elongate expandable member increases in outer diametrical size or cross-sectional area by a multiple of at least 5, at least 7.5, or at least 10 within a time period of 5 minutes, 4 minutes, 3 minutes, 2 minutes or less. Optionally, the elongate expandable member can be configured to expand slower such that a period of time longer than 5 minutes transpires before a final or appreciable expanded size or area is reached. As the elongate expandable member expands, the porous cover member also expands and provides longitudinal support to the elongate expandable member, thereby inhibiting mitigation of portions of the elongate expandable member. After the elongate expandable member expands an appreciable amount, the inner tubular member and the guidewire can be removed in operation 414.

In operation 416, excess portions of the elongate expandable member and the porous cover member can be cut and removed and, in operation 418, a proximal portion of the porous cover member can be secured to subcutaneous tissue. Securing the porous cover member to subcutaneous tissue can inhibit any migration of the assembly within the target vessel. Optionally, a separate tab component can be used to secure the position of the porous cover member to subcutaneous tissue. The elongate expandable member, the porous cover member, and the seal member can be configured to fully absorb in a period of one to six months, with tissue taking the place of the members. If deemed desirable by a caregiver, a pressure wrap or stocking can be temporarily applied around a patient's skin in the vicinity of the treated vessel portion(s).

FIG. 5 illustrates an elongate expandable member 516, a porous cover member 517, and a seal member 515 of an assembly located in, and occluding, a portion of a great saphenous vein 104 of a leg 102. In this example, the elongate expandable member 516 and the porous cover member 517 are placed between a point 506 near a medial side of the leg 102 and a point 508 near a junction between the great saphenous vein 104 and a femoral vein 112, at which the seal member 515 is positioned.

Initially disposed in a radially compressed configuration to ease insertion and even deployment, the elongate expandable member 516 and the porous cover member 517 can be configured to quickly expand upon removal of an outer member in situ. The elongate expandable member 516, when wetted within the vein 104, can expand from a first diametrical size or first cross-sectional area to a second larger diametrical size or second larger cross-sectional area and, in so doing, urge expansion of the surrounding porous cover member 517. In various examples, the second larger diametrical size or second larger cross-sectional area is at least 5 times or at least 10 times the first diametrical size or first cross-sectional area. In some examples, the second diametrical size or second cross-sectional area is substantially equal to a pre-wetted size or area of the member before being radially compressed and positioned between portions of the removable inner tubular member and the removable outer member.

Each of the elongate expandable member 516 and the porous cover member 517 can include a length of at least 10 cm and can have a vessel compliant outer surface. The elongate structures and compliant outer surfaces can provide a large contact surface with the walls of the vein 104 to occlude the conduit. With the conduit of the vein 104 occluded, blood previously flowing through the conduit will be rerouted to other network veins for its circulation. While the body's natural healing gradually permanently seals the treated vein, the porous cover member 517 inhibits migration of any portions of the elongate expandable member 516.

While discussions of FIG. 5 focus on occluding the great saphenous vein 104 via access at the knee level, the great saphenous vein 104 can also be accessed at a higher (e.g., jugular) level or a lower (e.g., near the ankle) level. During such access, any portion of the vein 104 existing between the ankle and the sapheno-femoral junction can be subjected to occlusion. Other veins in the leg that may be involved in the varicose vein condition, e.g., spider veins, can also be occluded, alternatively or in addition to the great saphenous vein 104.

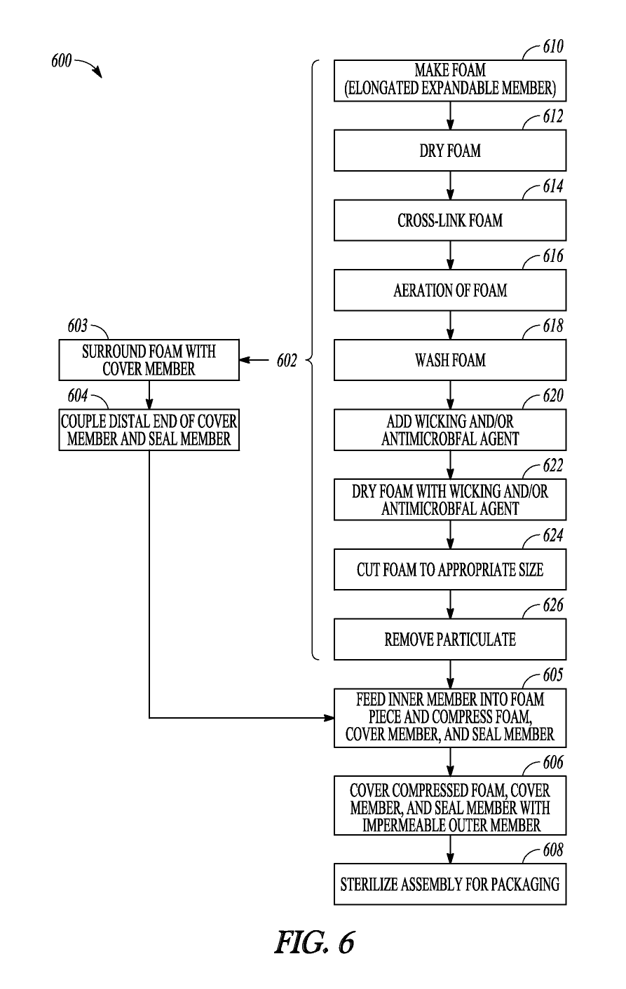

FIG. 6 illustrates a method 600 of manufacturing an assembly for occluding a vascular vessel, as constructed in accordance with at least one embodiment. The method can include, in varying orders, manufacturing an elongate expandable member 602, covering the elongate expandable member with a porous cover member 603, coupling a distal end portion of the porous cover member and a seal member 604, feeding a removable inner tubular member into the center portion of the elongate expandable member and radially compressing the elongate expandable member, the porous cover member, and, optionally, the seal member onto the removable inner tubular member 605, covering at least a portion of the radially compressed elongate expandable member, the porous cover member, and the seal member with a removable outer member 606, and sterilizing the assembly for packaging 608.

Manufacturing the elongate expandable member 602 can include creating a sponge- or foam-like matrix (e.g., sponge) structure having a relatively low density, large pore size, high degree of cross-linking, basic pH level, large radially compression ratio, and fast swell time when wetted.

In operation 610, a sheet of expandable gelatin can be treated to initiate the manufacture of the elongate expandable member 602. In some examples, the elongate expandable member can include treated reconstituted or naturally-derived collagenous materials to promote cellular growth within the member, thereby promoting permanent closure of an occluded passageway. Through proper treatment, the elongate expandable member can be configured to expand by at least about 5, at least about 6, at least about 7, at least about 8, at least about 9, at least about 10, and up to about 15 times its radially compressed diameter or cross-sectional area, or more. In some examples, the elongate expandable member is capable of expansion to its original, pre-compressed diameter or cross-sectional area. The magnitude of the expansion can be tailored by, among other things, varying the elongate expandable member's density, degree of cross-linking, sterilization method, dryness, and concentration of a wicking agent or pH adjuster. In an example, the gelatin is treated with a pH adjuster selected from hydrochloric acid, sodium hydroxide, or a buffer in a concentration resulting in the gelatin matrix having a pH greater than 5.7. In an example, the treated gelatin matrix has a density between 0.005 g/cm³ and 0.010 g/cm³ and exhibits relatively large pore sizes.

The treated gelatin can be dried sufficiently in operation 612 to stabilize the matrix. Drying of the gelatin matrix can involve high flow of dehumidified air, vacuum drying at ambient or elevated temperatures, or freeze drying. The drying procedure can reduce the liquid (e.g., water) content of the gelatin matrix to less than about 20% by weight, and more preferably, less than about 10% by weight.

Cross-linking can be used in operation 614 to impart desirable radially compression and expansion properties to the gelatin matrix. For example, cross-linking of a later compressed matrix can promote re-expansion of the matrix after implantation into a patient's vessel. The amount of added cross-linking within the gelatin matrix can be selected depending upon a desired treatment regime (e.g., occlusion duration or swell time for vessel fixation). In many examples, the gelatin matrix is cross-linked to complete an in situ expansion process over the course of minutes and prevent its degradation for at least 20 days, at least 30 days, and up to at least 90 days, or more. Cross-linking bonds can be initiated by the inclusion of formaldehyde or glutaraldehyde in vapor or liquid form, for example. Other cross-linking agents that can be used in vapor or liquid formulations include isothiocyanates, isocyanates, acyl azides, NHS Esters, aldehydes, epoxides, carbodiimides, anhydyrids, genipin, and combinations thereof. The amount of cross-linking can be determined using DSC testing, for example, and numerically reported as Lysine residuals. A smaller Lysine residuals percentage represents a higher degree of cross-linking. In an example, the gelatin matrix includes Lysine residuals of 1.5% or less. In operations 616 and 618, the cross-linked gelatin matrix can be aerated and washed to reduce residuals of formaldehyde or other cross-linking agents.

A formulation including a wicking or wetting agent can be made in operation 620 and added to the cross-linked gelatin matrix. The wicking agent can be a biocompatible substance that facilitates or enhances hydration and/or lubrication of the gelatin matrix when implanted in a target vessel. In an example, the wicking agent can be selected from a salt (e.g., sodium chloride) or a sugar. Other suitable wicking agents include polysaccharides, polyoxyalkylenes, glycerin, and water soluble polymers. Optionally, an antimicrobial agent can also be added to the gelatin matrix to destroy or interrupt microbial development and pathogenic actions. In an example, the antimicrobial agent can be selected from silver compounds, chlorhexidine, antibiotics, iodine-containing agents, and certain heavy metals. Experimental results have shown that the use of an antimicrobial agent in the form of a silver compound can result in a greater than 4 log reduction in bacterial contamination. The wicking formulation and optionally, the antimicrobial agent, can be dried into the gelatin matrix in operation 622. In an example, a freeze drying process is used in operation 622. The freeze drying can be performed at varying air pressures, matrix temperatures, and shelf temperatures for a period of days. Other suitable drying processes include air drying, vacuum drying, oven drying, and lyophilization. The drying procedure can reduce the liquid content of the gelatin matrix to less than about 10% by weight, and more preferably, less than about 1-2% by weight.

The cross-linked gelatin matrix including a wicking agent and optionally an antimicrobial agent can be cut (e.g., tore, grinded, sheared, etc.) to a desired size in operation 624. The cutting process can be manual or automated. In operation 636, particulate can be removed from the matrix using a vacuum or air brushing process and can complete the manufacturing of the elongate expandable member 602.

The elongate expandable member can be covered with the porous cover member in operation 603. The porous cover member can have a length longer than a length of the elongate expandable member such that excess cover member material is available at each end of the elongate expandable member. In operation 604, the excess cover member material adjacent the distal end of the elongate expandable member can be coupled with an outer surface of the seal member.

In operation 605, the removable inner tubular member can be fed into the center portion of the elongate expandable member, and the elongate expandable member, the porous cover member, and, optionally, the seal member can be compressed onto an outer surface of the removable inner tubular member. Compression forces can be applied so as to achieve a desired density or configuration, and can be applied in one, two, or three dimensions, including radially. When processed in this manner, upon removal of the compression force, the elongate expandable member and the porous cover member can be stabilized structurally and remain in a dense and compacted state until contacted with a liquid (e.g., body fluids) susceptible to absorption by the elongate expandable member matrix. The pores of the elongate expandable member can be retained at a volume substantially reduced from their maximum volume, but can return to a partially or fully expanded state when the matrix is wetted. In an example, the pre-compressed elongate expandable member can have a generally square shape. In an example, the radially compressed elongate expandable member can have a generally cylindrical shape with a generally circular cross section, and can have a diameter approximating that or smaller than that of an introducer sheath through which it is to be passed. In an example, the radially compression forces can cause a 10-to-1 diameter or cross-sectional area change of the elongate expandable member.

The removable outer member can be applied over the radially compressed elongate expandable member, porous cover member, and seal member in operation 606 to inhibit premature expansion when the assembly is introduced into a vessel. Finally, in operation 608, the assembly including the removable inner tubular member, the elongate expandable member, the porous cover member, the seal member, and the removable outer member can be sterilized for packaging. The sterilization process can be completed using one or more of irradiation (e.g., E-beam), gamma sterilization, ethylene oxide gas, or dry heat sterilization. Experimental results have shown that a combination of an E-beam process followed by a heat process can result in a greater than 4 log reduction in viral contamination.

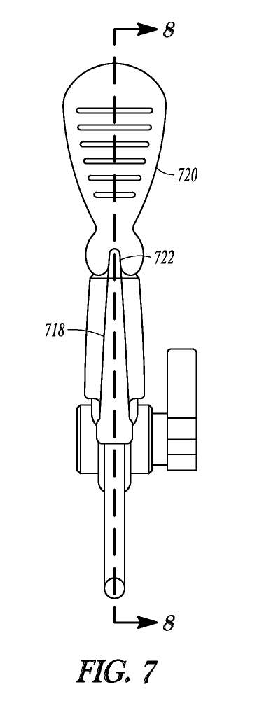

FIG. 7 illustrates a proximal end view of an assembly 700 for occluding a vascular vessel, as constructed in accordance with at least one embodiment. The assembly 700 can comprise a removable inner tubular member, a removable outer member, an elongate expandable member, a porous cover member, and an optional seal member. The elongate expandable member and the porous cover member can be positioned in a radially compressed form between portions of the removable inner tubular member and the removable outer member. The seal member can be positioned at a distal end of the elongate expandable member and partially surrounded by the removable outer member. The inner and outer members can each include a handle or hub 718 and 720, respectively, coupled to its proximal end. Adjacent inner surfaces of the handles or hubs 718 and 720 can form a snap-fit connection 722, which can be separated when desired by an operator.

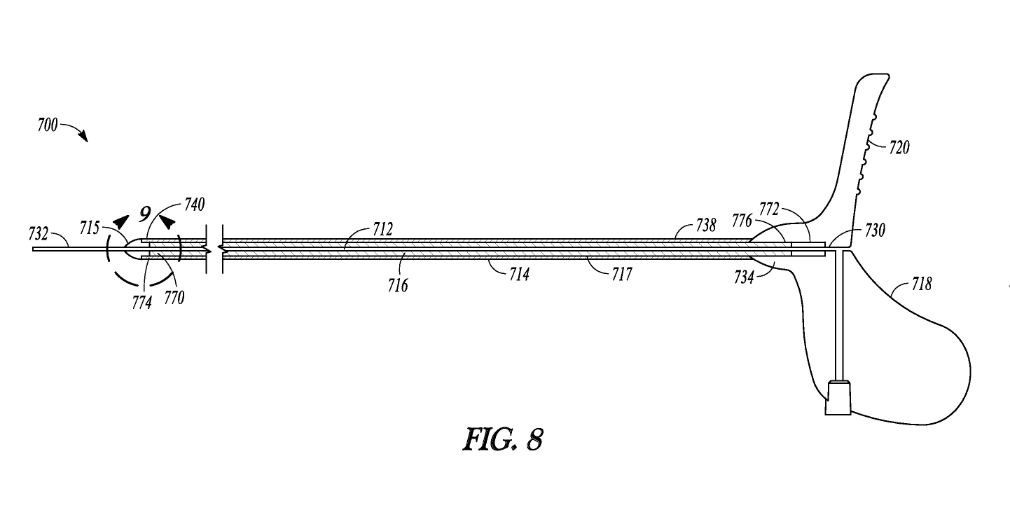

FIG. 8 illustrates a side cross-sectional view of the assembly 700, such as along line 8-8 of FIG. 7. Moving from inside-out, the assembly 700 can include the removable inner tubular member 712, the elongate expandable member 716 radially compressed onto an outer surface of the inner tubular member 712, the porous cover member 717 radially compressed onto an outer surface of the elongate expandable member 716, the optional seal member 715 positioned at a distal end of the elongate expandable member 716 and coupled with a distal end of the porous cover member 717, and the removable outer member 714 surrounding an outer surface of the porous cover member 717 and a portion of an outer surface of the seal member 715.

The removable inner tubular member 712 can extend from a proximal end 730 to a distal end 732 and can have a length longer than a length of the elongate expandable member 716, the porous cover member 717, and the removable outer member 714. The removable inner tubular member 712 can include a polyimide material having a tubular configuration for receiving a guidewire during a vessel introduction process. The proximal end 730 can be attached to a handle or hub 718 including an integrated blade 734. The blade 734 can be used to cut the removable outer member 714 in a proximal-to-distal direction. Optionally, a side-arm member can be attached to the proximal end 730 to provide access to an introduction lumen of the removable inner tubular member 712. An infusion of fluid into the introduction lumen by way of the side-arm member can function to flush the contents of the lumen. While various examples discussed in this document include a removable inner tubular member, the present inventors recognize that similar assemblies to those discussed can be created without the inclusion of the inner tubular member.

The removable outer member 714 can extend from a proximal end 738 to a distal end 740. The removable outer member 714 can be positioned such that its distal end 740 extends beyond a distal end of the elongate expandable member 716 but less than the distal end 732 of the removable inner tubular member 712. The removable outer member 714 can include an impermeable polytetrafluoroethylene or polyimide material and a configuration providing pushability or column strength to the assembly 700. The proximal end 738 can be attached to a handle or hub 720 and be movable in a direction away from the handle or hub 718 when an operator desires to cut and remove the outer member 714. The distal end 740 can be coupled with an outer surface of the seal member 715.

The elongate expandable member 716 can be positioned between and longitudinally sealed by the removable inner tubular member 712 and the removable outer member 714. The distal end 770 of the elongate expandable member 716 can be sealed by the seal member 715. The elongate expandable member 716 is initially deployed in a radially compressed configuration to facilitate its delivery through vasculature and within an introducer sheath. After reaching a desired implantation site, the outer member 714 can be removed, thereby allowing the elongate expandable member 716 to radially expand to an operative configuration in which the outer surface of the member 716 can engage surrounding vessel walls. Post-expansion, the elongate expandable member 716 can occlude the conduit of the vessel and prevent blood from flowing therethrough.

The porous cover member 717 can also be positioned between and longitudinally sealed by the removable inner tubular member 712 and the removable outer member 714. The porous cover member 717 extends from a proximal end 772 to a distal end 774 and surrounds the longitudinal outer surface of the elongate expandable member 716 to provide support thereto. The porous cover member 717 can expand from a compressed or unexpanded delivery configuration to a radially expanded deployment configuration through expansion of the elongate expandable member 716. The distal end 774 of the porous cover member 717 can extend beyond the distal end 770 of the elongate expandable member 716 and can be coupled with an outer surface of the seal member 715 at a location proximal to the distal end 740 of the removable outer member 714. The proximal end 772 of the porous cover member 717 can extend beyond a proximal end 776 of the elongate expandable member 716 and be used to suture the assembly 700 to subcutaneous tissue.

While the terms “compressed,” “unexpanded,” and “compacted” have been used to describe the elongate expandable member and the porous cover member as having small diameters or cross-sectional areas necessary for delivery to an implantation site, it is to be appreciated that the terms should not be used to imply that the members are under external pressure to provide the small diameters or cross-sectional areas; i.e., a “compressed,” “unexpanded,” or “compacted” member can be formed and subsequently naturally reside in the “compressed,” “unexpanded,” or “compacted” state until internally pressurized to expand through fluid absorption. Therefore, “compressed,” “unexpanded,” and “compacted” are intended only to imply that the elongate expandable member and the porous cover member are in a state of having small diameters or cross-sectional areas relative to expanded states.

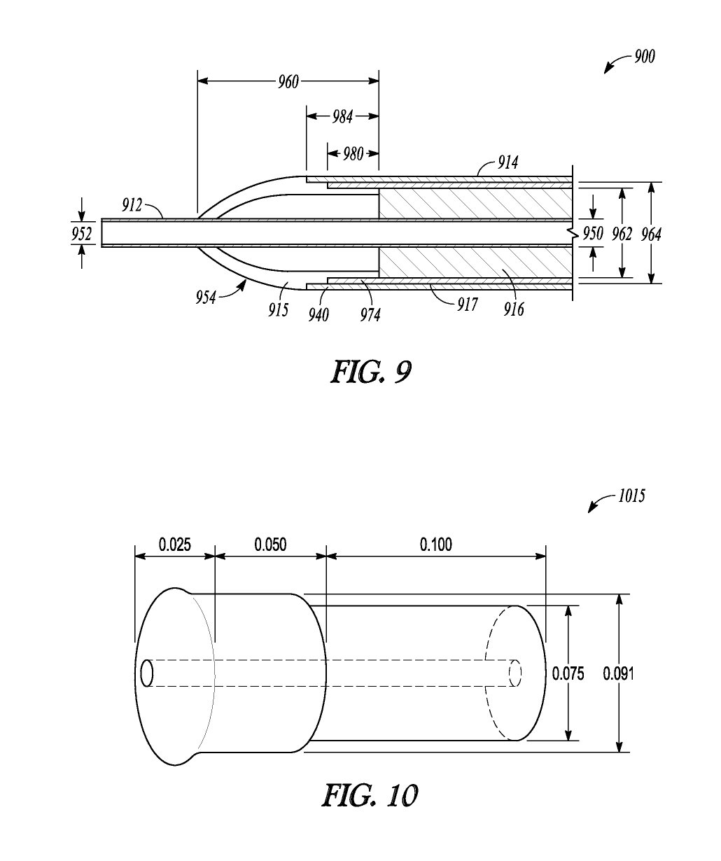

FIG. 9 illustrates a side cross-sectional view of a distal end portion of an assembly 900 for occluding a vascular vessel, as constructed in accordance with at least one embodiment. The assembly 900 can include a removable inner tubular member 912, an elongate expandable member 916, a porous cover member 917, a seal member 915, and a removable outer member 914.

The removable inner tubular member 912 can longitudinally extend beyond distal ends of the elongate expandable member 916, the porous cover member 917, and the removable outer member 914. In an example, the removable inner tubular member 912 can include an outer diameter 950 of about 0.023 in and an inner diameter 952 of about 0.022 in.

The seal member 915 can have a main body length 960, including a first portion 980 having a first diameter 962, a second portion 984 having a larger second diameter 964, and tapered radius of curvature 954. The main body length 960 can range from about 0.1 to about 0.3 in, such as about 0.175 in. The first portion 980 can have a length ranging from about 0.050 in to about 0.150 in, such as about 0.100 in, and the first diameter 962 can range from about 0.050 in to about 0.100 in, such as about 0.075 in. The second portion 984 can having a length ranging from about 0.075 in to about 0.200 in, such as about 0.150 in, and the second diameter 964 can range from about 0.075 in to about 0.095 in, such as about 0.088 in. The tapered radius of curvature 954 can range from about 0.100 in to 0.250 in, such as about 0.175 in.

A distal end 974 of the porous cover member 917 and a distal end 940 of the removable outer member 914 can be coupled with outer surface portions of the seal member 915, such as outer surface portions of the first portion 980 and the second portion 984, respectively. As a result, the first portion 980 of the seal member 915 can be surrounded by the distal end 974 of the porous cover member 917, and the second portion 984 of the seal member 915 can be surrounded by the distal end 940 of the removable outer member 914. The coupling of the seal member 915 with each of the porous cover member 917 and the removable outer member 914 can include one or more welds (e.g., RF welds), adhesive, glue or other bonding means.

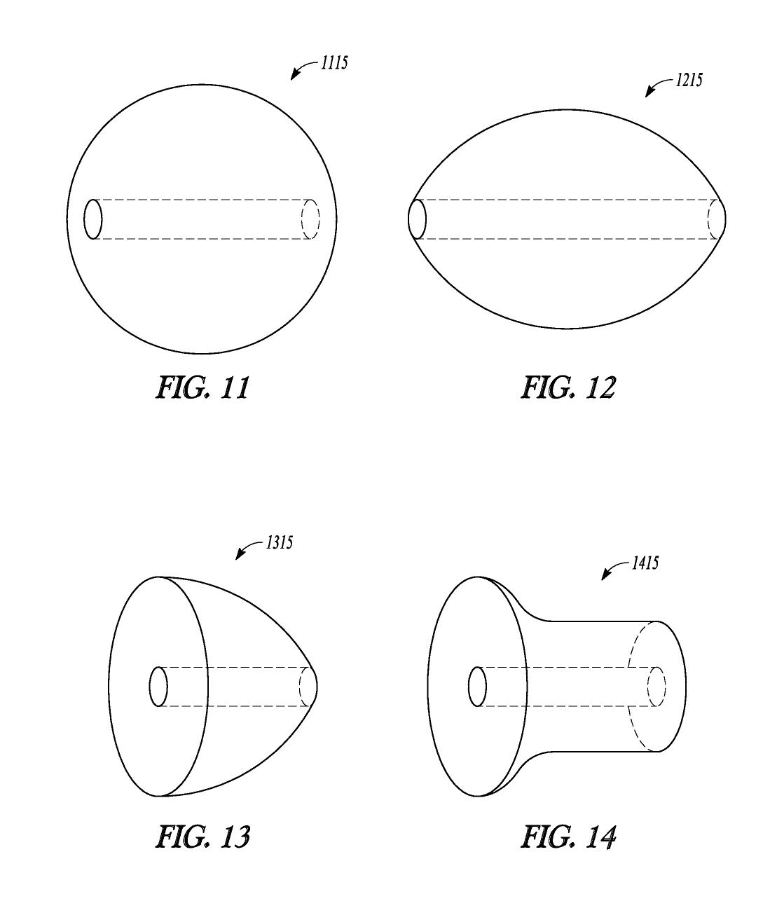

FIGS. 10-14 illustrate various shapes of seal members 1015 (champagne cork shape), 1115 (spherical shape), 1215 (football shape), 1315 (space shuttle shape), and 1415 (plug shape) that can be positioned at a distal end of an elongate expandable member and coupled with a removable outer member to inhibit the elongate expandable member from expanding until an operator-elected instant. Optionally, the seal members 1015, 1115, 1215, 1315, and 1415 can include an x-ray visible material to guide introduction of the present assembly to a desired depth with a target vascular vessel.

Each seal member can include a rounded or tapered tip, designed to be atraumatic, an axial extension for coupling with one or both of the porous cover member or the removable outer member, and a lumen through which a removable inner tubular member can extend for receipt of a guidewire. The seal member can be removed from the target vascular vessel after implantation of the elongate expandable member and the porous cover member or, alternatively, composed of one or more bioabsorbable or biodegradable materials (e.g., polylactide or a polyglycolide) or a more permanent material and left in place.

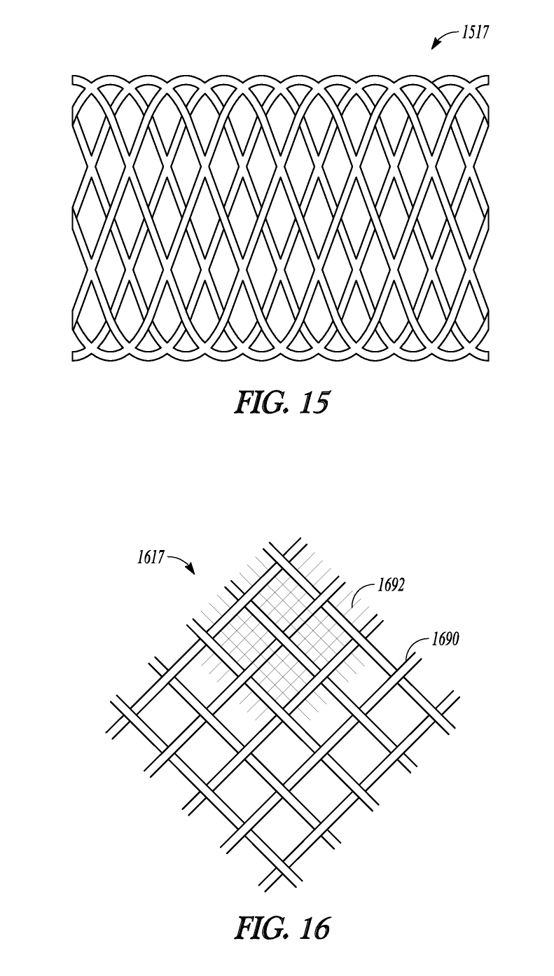

FIGS. 15 and 16 illustrate porous cover members 1517 and 1617, respectively, which can be positioned around an outer surface of an elongate expandable member to support its longitudinal integrity. The porous cover member 1517 shown in FIG. 15 includes a braided structure. The porous cover member 1617 shown in FIG. 16 includes a woven structure having primary filaments 1690 and secondary filaments 1692. The secondary filaments 1962 can, for example, be smaller, more elastic or have a faster absorption rate than the primary filaments 1690.

Porous cover members can have a variety of configurations provided that such configurations allow fluid absorption by the enclosed elongate expandable member and have the ability to expand upon expansion of the elongate expandable member. In addition to the braided and woven structures shown in FIGS. 15 and 16, the porous cover members can include helically wound strands, ring members, tube members, struts having a zigzag pattern, filaments having a sinusoidal shape, or knitted filaments. A porous cover member structure and configuration can be chosen to facilitate maintenance of the elongate expandable member in the vessel following implantation.

The porous cover members can also have a variety of sizes. The exact size chosen will depend on several factors, including the desired delivery technique, the nature of the target vessel to be treated, and the size of the vessel. In some examples, the porous cover member can have a length in the range from about 3 cm to about 90 cm, usually being from about 20 cm to about 60 cm, for vascular applications. The expanded diameter of the porous cover member can be in the range from about 2 mm to about 30 mm, preferably being in the range from about 10 mm to about 25 mm for vascular applications.

The size and number of yarns or filaments composing the porous cover members can be determined in such a way that the period needed for absorption or degradation of the occlusion device is greater than or equal to the period of natural absorption of the treated vessel once the occlusion has been performed.

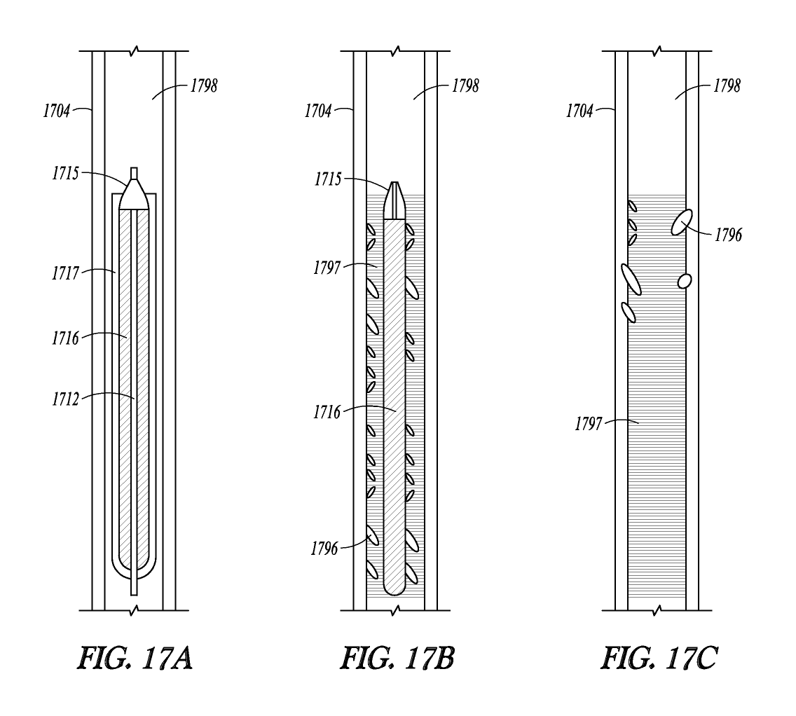

FIGS. 17A-17C schematically illustrate the gradual absorption and/or degradation of an elongate expandable member 1716, a porous cover member 1717, and an optional seal member 1715 within a portion of a great saphenous vein 1704 post-implant.

In accordance with the method teachings described in association with FIG. 4, an assembly—including a removable inner tubular member 1712, a removable outer member, the elongate expandable member 1716, the porous cover member 1717, and the seal member 1715—can be introduced within a patient's body via percutaneous access through the skin and into the portion of the great saphenous vein 1704 to be treated. The assembly can be advanced intravascularly through the vein 1704 and positioned proximal of the sapheno-femoral junction until the portion to be treated has been reached or traversed by the assembly. An echogenic or radio-opaque marker can optionally be disposed near or at a distal end of the assembly to facilitate visualization and positioning of the assembly within the vein 1704 via, e.g., ultrasound or fluoroscopy. Although a single assembly is illustrated, one or more assemblies positioned in series (e.g., end-to-end) can be used.

Once desirably positioned proximate to the vein portion to be treated, the outer tubular member can be removed such that the porous cover member 1717 and the elongate expandable member 1716 are exposed to fluid 1798 within the vein 1704. The elongate expandable member 1716 is now free to absorb the fluid 1798 and radially expand, along with the porous cover member 1717, to occlude the conduit of the vein 1704, as illustrated in FIG. 17A. A sponge- or foam-like structure of the elongate expandable member 1716 can ensure reliable occlusion of the vein's conduit.

After the elongate expandable member expands an appreciable amount, the removable inner tubular member 1712 can be removed and a proximal portion of the porous cover member 1717 can be secured subcutaneously, as illustrated in FIG. 17B. Securing the porous cover member subcutaneously inhibits any migration of the assembly within the vein 1704. As the elongate expandable member 1716 continues to remain within the vein 1704, the vein wall 1799 begins the formation of granulation tissue 1796. Granulation tissue 1796 can include capillaries, fibroblasts, and a plurality of cells.

The elongate expandable member 1716, the porous cover member 1717, and the seal member 1715 remain within the vein 1704 in contact with the vein wall 1799 to continue and maintain the cellular response until the members are fully absorbed and/or degraded, leaving behind remodeled tissue 1797. FIG. 17C illustrates an almost completely remodel vein and the complete absorption and/or degradation of the elongate expandable member 1716, the porous cover member 1717, and the seal member 1715.

Experimental Results:

Laboratory experiments were conducted to help quantify properties of example elongate expandable members, as conceived by the present inventors. In these experiments, elongate expandable members were manufactured using the teachings described in association with FIG. 6. Each elongate expandable member was designed to occlude a vascular vessel (e.g., a varicose vein) by occupying the vessel's full cross-section and exerting sufficient radial pressure and friction on surrounding vessel walls to remain in place even when subjected to vessel pressures.

Further laboratory experiments were conducted to verify that the elongate expandable member exerted sufficient radial expansion when wetted to expand the porous cover member surrounding its longitudinal outer surface. These laboratory experiments also verified that the porous cover member effectively contained the elongate expandable member and inhibited migration of elongate expandable member particles.

### 1. Experiment 1:

In this experiment, Lysine residuals of an elongate expandable member were explored relative to Lysine residuals of Pfizer's GELFOAM® product. The Lysine residuals were completed by hydrolysis of a sample sponge matrix from each product and running the sample on mass spectrometry.

TABLE 1
Experimental results showing that the present elongate
expandable member exhibits lower Lysine residuals than
a commercially available foam product.

| Product | Lysine Residuals (%) |
|---|---|
| Elongate expandable member | 1.1 |
| Pfizer's GELFOAM ® product | 1.7 |

### 2. Experiment 2:

In this experiment, the pH of an elongate expandable member including 0.03% sodium chloride and an elongate expandable member including 0.03% sodium chloride and 100 parts per million (ppm) of silver were explored relative to the pH of Pfizer's GELFOAM® product.

TABLE 2
Experimental results showing that the present elongate
expandable members include a higher (more basic)
pH than a commercially available foam product.

| Product | pH |
|---|---|
| Elongate expandable member including | 6.55 |
| 0.03% sodium chloride and 100 ppm of | |
| silver | |
| Elongate expandable member including | 5.93 |
| 0.03% sodium chloride | |
| Pfizer's GELFOAM ® product (at | 4.64 |
| saturation point) | |

### 3. Experiment 3:

In this experiment, the pepsin digestion of an elongate expandable member was explored relative to the pepsin digestion of Pfizer's GELFOAM® product and the United States Pharmacopeia (USP) sponge requirement. Pepsin is a digestive protease that degrades gelatin. The length of time it takes to degrade a wetted gelatin sponge relates to a degree of cross-linking present in a sample.

TABLE 3
Experimental results showing that the present elongate expandable
member includes a longer pepsin digestion time than a commercially
available foam product and the USP sponge requirement.

| Product | Time |
|---|---|
| Elongate expandable member | greater than 3 days |
| Pfizer's GELFOAM ® product | approximately 15 minutes |
| USP sponge requirement | less than or equal to 75 minutes |

### 4. Experiment 4:

In this experiment, a compression ratio of an elongate expandable member was explored relative to a compression ratio of Pfizer's GELFOAM® product. The compression ratio was calculated by comparing a sample sponge size for each product prior to compression and after compression. A standardized sample size of each product was prepared (1 cm×1 cm×5 cm) and each product was compressed for 15 seconds at 100 pounds per square inch (psi) using a Machine Solutions stent crimper.

TABLE 4
Experimental results showing that the present elongate
expandable member can be compressed to a smaller outer
diameter than a commercially available foam product.

| Product | Compressed Diameter (inches) |
|---|---|
| Elongate expandable member | 0.047 |
| Pfizer's GELFOAM ® product | 0.052 (approximately 11% larger than |
| | the elongate expandable member) |

### 5. Experiment 5:

In this experiment, a swell ratio and time of an elongate expandable member was explored relative to a swell ratio and time of Pfizer's GELFOAM® product. The swell ratio is defined as the comparison of a volume for a pre-wetted compressed sponge of each product to the post-wetted sponge in 37 degree Celsius saline.

TABLE 5
Experimental results showing that the present elongate expandable
member includes a higher swell ratio and lower swell time than
a commercially available foam product.

| | | | | | Time to |
|---|---|---|---|---|---|
| | Pre- | Post- | Post- | % of | Reach |
| | Wetted | Compression | Wetted | Volume | Stable Size |
| Elongate | 1 cm × | 0.047 in | 1 cm × | 100% | less |
| expandable | 1 cm × | diameter | 1 cm × | | than 4 |
| member | 5 cm | | 5 cm | | seconds |
| Pfizer's | 1 cm × | 0.052 in | 0.84 cm × | 67% | 10 |
| GELFOAM ® | 1 cm × | diameter | 0.86 cm × | | minutes |
| product | 5 cm | | 4.66 cm | | |

Closing Notes:

Over 40 million people in the U.S. alone have varicose veins and suffer from the aching, pain, leg heaviness and swelling, fatigue, and aesthetic issues associated with the disease. Advantageously, the present assemblies and methods provide a varicose vein treatment that is associated with minimal patient discomfort and a minimal risk of vessel perforation, does not require an investment in capital equipment or insulation, and can be completed relatively quickly and easily. Treatment components are mechanically stable, resist migration, and leave behind a cosmetically pleasing implant.

Upon removal of an outer member of an assembly, a radially compressed elongate expandable member and a surrounding porous cover member are allowed to radially expand inside a target vein by absorbing fluid, providing occlusion of the vein. Migration of the assembly can be inhibited by securing a proximal portion of the porous cover member subcutaneously. An optional seal member, positioned at a distal end of the elongate expandable member and the porous cover member, can preserve a dry state and inhibit expansion of the elongate expandable member and porous cover member until an operator-elected time period.

The above Detailed Description includes references to the accompanying drawings, which form a part of the Detailed Description. The Detailed Description should be read with reference to the drawings. The drawings show, by way of illustration, specific embodiments in which the present assemblies and methods can be practiced. These embodiments are also referred to herein as “examples.”

The above Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, an assembly can comprise a removable outer member optionally including an impermeable material, an elongate expandable member including a sponge- or foam-like structure, and a porous cover member surrounding an outer surface of the elongate expandable member. The elongate expandable member and the porous cover member can be positioned in a radially compressed form within the removable outer member. The elongate expandable member can be configured to expand the porous cover member and occlude a vascular vessel following removal of the removable outer member.

In Example 2, the assembly of Example 1 can optionally further comprise a seal member positioned at a distal end of the elongate expandable member.

In Example 3, the assembly of Example 2 is optionally configured such that the seal member includes a first portion having a first diameter and a second portion having a larger second diameter.

In Example 4, the assembly of Example 3 is optionally configured such that the first portion of the seal member is surrounded, at least in part, by the porous cover member.

In Example 5, the assembly of any one of Examples 3 or 4 is optionally configured such that the second portion of the seal member is surrounded, at least in part, by the removable outer member.

In Example 6, the assembly of any one or any combination of Examples 3-5 is optionally configured such that the second portion of the seal member tapers from the second diameter to a distal tip.

In Example 7, the assembly of any one or any combination of Examples 2-6 is optionally configured such that the seal member is coupled at one or more locations to the porous cover member or the removable outer member.

In Example 8, the assembly of any one or any combination of Examples 2-7 is optionally configured such that the seal member includes one or both of a polylactide material or a polyglycolide material.

In Example 9, the assembly of any one or any combination of Examples 2-8 optionally further comprises a removable inner tubular member positioned within the elongate expandable member, and is optionally configured such that the seal member includes a lumen coaxial with, and longitudinally adjacent to, a lumen defined by the removable inner tubular member.

In Example 10, the assembly of Example 9 is optionally configured such that each lumen is sized and shaped to receive a guidewire.

In Example 11, the assembly of any one or any combination of Examples 1-10 is optionally configured such that the porous cover member is configured to support a longitudinal integrity of the elongate expandable member.

In Example 12, the assembly of any one or any combination of Examples 1-11 is optionally configured such that the porous cover member includes a knitted structure.

In Example 13, the assembly of any one or any combination of Examples 1-11 is optionally configured such that the porous cover member includes a woven structure.

In Example 14, the assembly of any one or any combination of Examples 1-11 is optionally configured such that the porous cover member includes a braided structure.

In Example 15, the assembly of any one or any combination of Examples 1-14 is optionally configured such that the porous cover member includes a bioabsorbable material.

In Example 16, the assembly of Example 15 is optionally configured such that the bioabsorbable material includes one or both of a polylactide material or a polyglycolide material.

In Example 17, the assembly of any one or any combination of Examples 1-16 is optionally configured such that the elongate expandable member includes a gelatin material or a collagen material having a degree of vapor cross-linking characterized by Lysine residuals of 1.5% of less.

In Example 18, the assembly of any one or any combination of Examples 1-17 is optionally configured such that the elongate expandable member and the porous cover member are configured to expand, when wetted, from a radially compressed first diametrical size or first cross-sectional area to a second larger diametrical size or second larger cross-sectional area, which is at least 5 times the first diametrical size or cross-sectional area.

In Example 19, the assembly of Example 18 is optionally configured such that the elongate expandable member and the porous cover member include a length of at least 10 centimeters, and wherein each member expands in situ from the first diametrical size or first cross-sectional area to the second larger diametrical size or second larger cross-sectional area within a time period of 5 minutes or less.

In Example 20, a method can comprise inserting an elongate expandable member and a porous cover member into a vascular vessel. The elongate expandable member and the porous cover member can be radially compressed, optionally about a removable inner tubular member, enclosed around their respective outer surfaces by a removable outer member, and optionally distally sealed by a seal member. The elongate expandable member and the porous cover member can be advanced through the vascular vessel by guiding a lumen of the inner tubular member along a guidewire. The outer member can be removed to allow the elongate expandable member and the porous cover member to expand from a radially compressed first diametrical size or first cross-sectional area to a second larger diametrical size or second larger cross-sectional area and occlude the vascular vessel.

In Example 21, the method of Example 20 can optionally be configured such that inserting the elongate expandable member and the porous cover member, enclosed around their respective outer surfaces by the removable outer member and optionally distally sealed by the seal member, into the vascular vessel includes inhibiting expansion of a portion of the elongate expandable member until an operator-elected time period.

In Example 22, the method of any one of Examples 20 or 21 can optionally be configured such that inserting the elongate expandable member and the porous cover member into the vascular vessel includes inserting an elongate expandable member and a porous cover member having a length of at least 10 centimeters into a great saphenous vein or a lesser saphenous vein.

In Example 23, the method of any one or any combination of Examples 20-22 can optionally be configured such that removing the outer member includes separating an engagement between an outer surface of the seal member and an inner surface of the removable outer member and preserving an engagement between the outer surface of the seal member and an inner surface of the porous cover member.

In Example 24, the method of any one or any combination of Examples 20-23 can optionally be configured such that allowing the elongate expandable member and the porous cover member to expand to the second larger diametrical size or second larger cross-sectional area includes maintaining integrity of the elongate expandable member by way of the porous cover member.

In Example 25, the method of any one or any combination of Examples 20-24 can optionally be configured such that allowing the elongate expandable member and the porous cover member to expand includes increasing an outer diametrical size of the elongate expandable member a multiple of at least 5 within a time period of 5 minutes or less.

In Example 26, the method of any one or any combination of Examples 20-25 can optionally further comprise inhibiting migration of the elongate expandable member by securing a portion of the porous cover member to subcutaneous tissue.

In Example 27, the method of any one or any combination of Examples 20-26 can optionally further comprise removing and discarding excess elongate expandable member following expansion of the elongate expandable member and the porous cover member from the first diametrical size or first cross-sectional area to the second larger diametrical size or second larger cross-sectional area.

In Example 28, the method of any one or any combination of Examples 20-27 can optionally further comprise removing the inner tubular member at a time after the elongate expandable member and the porous cover member expand from the first diametrical size or first cross-sectional area.

In Example 29, the method of any one or any combination of Examples 20-28 can optionally further comprise promoting tissue in-growth into the elongate expandable member or the porous cover member by allowing for the release a drug, stored in the elongate expandable member, into a wall of the vascular vessel.

In Example 30, the method of any one or any combination of Examples 20-28 can optionally further comprise promoting tissue in-growth into the elongate expandable member or the porous cover member by allowing for the release a drug, stored in the porous cover member, into a wall of the vascular vessel.

In Example 31, the method of any one or any combination of Examples 20-30 can optionally further comprise inhibiting the colonization of microorganisms in the elongate expandable member or the porous cover member by allowing for the release of an antimicrobial agent stored in one or both of the elongate expandable member or the porous cover member.

In Example 32, the assembly or method of any one or any combination of Examples 1-31 is optionally configured such that all elements or options recited are available to use or select from.

Certain terms are used throughout this patent document to refer to particular features or components. As one skilled in the art will appreciate, different persons may refer to the same feature or component by different names. This patent document does not intend to distinguish between components or features that differ in name but not in function.

For the following defined terms and numeric values, certain definitions shall be applied, unless a different definition is given elsewhere in this patent document.

The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” refers to a range of numbers that one of skill in the art would consider equivalent to the recited value (e.g., having the same function or result). In many instances, the term “about” can include numbers that are rounded to the nearest significant figure. The recitation of numerical ranges by endpoints includes all numbers and sub-ranges within that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The term “patient” is intended to include mammals, such as for human or veterinary applications.

Various beneficial features of the present assemblies and methods are described in context of their relationship in use with a patient's anatomy. For the purposes of providing a clear understanding, the terms “proximal” and “proximally” should be understood to mean portions of an assembly relatively closer to an operator during use of the assembly, and the terms “distal” and “distally” should be understood to mean portions of the assembly relatively further away from the operator during use of the assembly.

The scope of the invention should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, kit or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. An assembly, comprising:
an elongate expandable member including a sponge- or foam-like structure; and
a porous cover member surrounding an outer surface of the elongate expandable member,
the elongate expandable member and the porous cover member positioned in a radially compressed form within a removable outer member, the elongate expandable member configured to expand the porous cover member and occlude a vascular vessel following removal of the removable outer member.

2. The assembly of claim 1, further comprising a seal member positioned at a distal end of the elongate expandable member.

3. The assembly of claim 2, wherein the seal member is coupled at one or more locations to the porous cover member or the removable outer member.

4. The assembly of claim 2, wherein the seal member includes one or both of a polylactide material or a polyglycolide material.

5. The assembly of claim 1, further comprising a removable inner tubular member positioned within the elongate expandable member, the removable inner tubular member defining a lumen sized and shaped to receive a guidewire.

6. The assembly of claim 1, wherein the porous cover member is configured to support a longitudinal integrity of the elongate expandable member.

7. The assembly of claim 6, wherein the porous cover member includes a knitted structure.

8. The assembly of claim 6, wherein the porous cover member includes a woven structure.

9. The assembly of claim 6, wherein the porous cover member includes a braided structure.

10. The assembly of claim 1, wherein the porous cover member includes a bioabsorbable material.

11. The assembly of claim 10, wherein the bioabsorbable material includes one or both of a polylactide material or a polyglycolide material.

12. The assembly of claim 1, wherein the elongate expandable member includes a gelatin material or a collagen material having a degree of vapor cross-linking characterized by Lysine residuals of 1.5% of less.

13. The assembly of claim 1, wherein the elongate expandable member and the porous cover member are configured to expand, when wetted, from a radially compressed first diametrical size or first cross-sectional area to a second larger diametrical size or second larger cross-sectional area, which is at least 5 times the first diametrical size or first cross-sectional area.

14. The assembly of claim 13, wherein the elongate expandable member and the porous cover member include a length of at least 10 centimeters, and wherein each member expands in situ from the first diametrical size or first cross-sectional area to the second larger diametrical size or second larger cross-sectional area within a time period of 5 minutes or less.

15. A method, comprising:
inserting an elongate expandable member and a porous cover member, both of which are radially compressed and enclosed around their respective outer surfaces by a removable outer member, into a vascular vessel;
advancing the elongate expandable member and the porous cover member through the vascular vessel; and
removing the removable outer member, including allowing the elongate expandable member and the porous cover member to expand from a radially compressed first diametrical size or first cross-sectional area to a second larger diametrical size or second larger cross-sectional area, and occlude the vascular vessel.

16. The method of claim 15, wherein inserting the elongate expandable member and the porous cover member, enclosed around their respective outer surfaces by the removable outer member, into the vascular vessel includes inhibiting expansion of a portion of the elongate expandable member until an operator-elected time period.

17. The method of claim 15, wherein inserting the elongate expandable member and the porous cover member into the vascular vessel includes inserting an elongate expandable member and a porous cover member having a length of at least 10 centimeters into a great saphenous vein or a lesser saphenous vein.

18. The method of claim 15, wherein allowing the elongate expandable member and the porous cover member to expand to the second larger diametrical size or second larger cross-sectional area includes maintaining integrity of the elongate expandable member by way of the porous cover member.

19. The method of claim 15, wherein allowing the elongate expandable member and the porous cover member to expand includes increasing an outer diametrical size of the elongate expandable member a multiple of at least 5 within a time period of 5 minutes or less.

20. The method of claim 15, further comprising inhibiting migration of the elongate expandable member by securing a portion of the porous cover member subcutaneously.

21. The method of claim 15, further comprising removing and discarding excess elongate expandable member following expansion of the elongate expandable member and the porous cover member from the first diametrical size or first cross-sectional area to the second larger diametrical size or second larger cross-sectional area.

22. The method of claim 15, further comprising promoting tissue in-growth into the elongate expandable member or the porous cover member by allowing for the release a drug, stored in one or both of the elongate expandable member or the porous cover member, into a wall of the vascular vessel.

23. The method of claim 15, further comprising inhibiting the colonization of microorganisms in the elongate expandable member or the porous cover member by allowing for the release of an antimicrobial agent stored in one or both of the elongate expandable member or the porous cover member.
